(19) United States
(12) Patent Application Publication
(10) Pub. No.: US 2017/0027771 A1
(43) Pub. Date: Feb. 2, 2017

(54) SHAPED ABSORBENT INSERT AND CONTAINMENT SYSTEM

(71) Applicant: Kimberly-Clark Worldwide, Inc., Neenah, WI (US)

(72) Inventors: Anita M. McLean, Sandy Springs, GA (US); Stacy E. Evenson, Neenah, WI (US)

(73) Assignee: Kimberly-Clark Worldwide, Inc., Neenah, WI (US)

(21) Appl. No.: 15/102,590

(22) PCT Filed: May 20, 2015
(86) PCT No.: PCT/US15/31707

§ 371 Date: Jun. 8, 2016

Related U.S. Application Data
(60) Provisional application No. 62/004,390, filed on May 29, 2014.

(51) Int. Cl. A61F 13/534; A61F 13/539; A61F 13/496; A61F 13/505; A61F 13/15

(52) U.S. Cl. CPC A61F 13/53409; A61F 13/505; A61F 13/15203; A61F 13/496; A61F 13/539; A61F 13/534; A61F 2013/15463; A61F 2013/15406; A61F 2013/53991; A61F 2013/53908; A61F 2013/5307

(57) ABSTRACT

A discrete absorbent insert and containment system including an absorbent core is disclosed. The absorbent insert includes a first sheet, a second sheet in facing relation with the first sheet, and an absorbent core positioned between the first sheet and the second sheet. The absorbent core defines a core perimeter and the first sheet and the second sheet extend beyond the core perimeter and are joined together to form a perimeter seal. A central section of an absorbent core is positioned between a first end section and a second end section. Both the first end section and the second end section comprise an arc extending at least 185 degrees. The first end section defines a maximum width of at least 120 mm, the central section defines a maximum width of less than 115 mm, and the second end section defines a maximum width of at least 120 mm.

### RELATED APPLICATION DATA

The present application claims priority to U.S. Provisional Application No. 62/004,390 entitled “SHAPED ABSORBENT INSERT AND CONTAINMENT SYSTEM” filed on May 29, 2014, in the names of Anita M. Gilgenbach et al., the contents of which are hereby incorporated by reference in a manner consistent with the present application.

### BACKGROUND OF THE INVENTION

This application is a continuation-in-part application claiming priority from presently copending U.S. Application No. 62/004,390 entitled “SHAPED ABSORBENT INSERT AND CONTAINMENT SYSTEM” filed on May 29, 2014, in the names of Anita M. Gilgenbach et al.

While disposable absorbent garments offer a convenient way to manage bedwetting, many enuretic families are not satisfied with these products because they are viewed as “diaper-like” by the user who wants to wear conventional underwear. As such, many improvements have been made to disposable absorbent garments to make them more underwear-like. For example, gender specific graphics have been added and softer nonwoven materials have been utilized. However, many potential users still seek a product that looks and feels more like conventional underwear but is still able to help manage bedwetting. Thus, there remains a need for a pant that is underwear-like yet helps to contain urine insult.

### SUMMARY OF THE INVENTION

In one embodiment, the present invention provides a discrete absorbent insert. The absorbent insert includes a first sheet, a second sheet in facing relation with the first sheet, and an absorbent core positioned between the first sheet and the second sheet. The absorbent core defines a core perimeter and the first sheet and the second sheet extend beyond the core perimeter and are joined together to form a perimeter seal. The absorbent core defines a longitudinal direction, a first end section, a second end section, and a central section. The central section is positioned between the first end section and the second end section. Both the first end section and the second end section comprise an arc extending at least 185 degrees. The first end section defines a maximum width of at least 120 mm, the central section defines a maximum width of less than 115 mm, and the second end section defines a maximum width of at least 120 mm.

In another embodiment, the absorbent core defines an absorbent core length as measured in the longitudinal direction. The first end section defines a first end section length that is 25-30% the absorbent core length. The second end section defines a second end section length that is 25-30% the absorbent core length. The central section defines a central section length that is 40-50% the absorbent core length. Both the first end section and the second end section may define a circular arc extending at least 270 degrees wherein the first end section defines a maximum width of at least 150 mm, the central section defines a maximum width of less than 100 mm, and the second end section defines a maximum width of at least 150 mm.

In some embodiments of this aspect, the discrete absorbent insert includes a first intake material positioned between the first sheet and the absorbent core. In some embodiments, the absorbent insert includes a second intake material positioned between the absorbent core and the second sheet.

In some embodiments of this aspect, the discrete absorbent insert is folded and individually wrapped in a wrapper. In some embodiments, the discrete absorbent insert is folded a first time such that a first portion of the first sheet is in facing relation with a second portion of the first sheet and is folded a second time such that a third portion of the first sheet is in facing relation with a first portion of the second sheet.

In some embodiments of this aspect, the first sheet is joined with the second sheet at the perimeter seal with adhesive and the perimeter seal has a width of at least 20 mm. In some embodiments, the first sheet is joined with the second sheet at the perimeter seal with an ultrasonic bond and the perimeter seal has a width of at least 10 mm.

In some embodiments, the absorbent insert includes an absorbent capacity of at least 600 g. The absorbent core may have at least 40% superabsorbent. In some embodiments, the absorbent core has at least 15 grams fluff and at least 15 grams superabsorbent. In some embodiments, the intake layer has a basis weight of at least 85 gsm.

In some embodiments of this aspect, the discrete absorbent insert is folded a first time such that a first portion of the first sheet is in facing relation with a second portion of the first sheet and is folded a second time such that a third portion of the first sheet is in facing relation with a first portion of the second sheet.

In some embodiments of this aspect, the discrete absorbent insert is devoid of fasteners and is devoid of containment flaps.

In another embodiment, a containment system including both a permanently closed containment pant and an absorbent insert is disclosed. The containment pant includes an elastically extensible chassis defining a waist opening, a first leg opening, and a second leg opening, the waist opening comprising a front waist region joined with a back waist region, and a sling positioned within the chassis and being joined to the front waist region and the back waist region. The sling is defined by a fluid-impervious base sheet and a containment flap joined with the base sheet to create a fluid-impervious pouch. The fluid-impervious pouch defines a pouch floor having a longitudinal direction, a first end section, a second end section, and a central section extending between the first end section and the second end section, wherein the first end section, the second end section, and the central section define equal lengths in the longitudinal direction and together define a pouch floor length. The first end section defines a maximum width of at least 145 mm, the central section defines a maximum width of less than 115 mm, and the second end section defines a maximum width of at least 145 mm. The containment system includes a discrete absorbent insert. The absorbent insert includes a first sheet, a second sheet in facing relation with the first sheet, and an absorbent core positioned between the first sheet and the second sheet. The absorbent core defines a core perimeter and the first sheet and the second sheet extend beyond the core perimeter and are joined together to form a perimeter seal. The absorbent core defines a longitudinal direction, a first end section, a second end section, and a central section. The central section is positioned between the first end section and the second end section. Both the first end section and the second end section comprise an arc extending at least 185 degrees. The first end section defines a maximum width of at least 120 mm, the central section defines a maximum width of less than 100 mm, and the second end section defines a maximum width of at least 120 mm.

### BRIEF DESCRIPTION OF THE DRAWINGS

FIG. 1 representatively illustrates a front perspective view of an exemplary containment pant of the present invention.

FIGS. 2 and 3 representatively illustrate side perspective views of the fully exemplary containment pant that is partially severed to illustrate internal structure.

FIG. 4 representatively illustrates a top plan view of component parts of another exemplary pouch and sling of the present invention

FIG. 5 representatively illustrates a fully constructed sling made from the component parts of FIG. 4.

FIG. 6 representatively illustrates a top plan view of an exemplary absorbent insert of the present invention with portions cut away to illustrate underlying structure.

FIG. 7 representatively illustrates a cross sectional view of the absorbent insert of FIG. 7 taken along the line C-C.

### DETAILED DESCRIPTION OF THE DRAWINGS

The containment pant of the present invention provides a durable outer chassis and an inner pouch for containing a disposable absorbent insert. In some embodiments, the containment pant is provided in a permanently closed condition. As used herein, the term “permanently closed” refers to a pant that is provided in a condition that is adapted to be only pulled on or pulled off like conventional underwear and is distinguished from pants that have refastenable fasteners and diapers that are provided in an open condition and include fasteners for securing the diapers around the body of the wearer. Suitable containment pants are also described in patent application U.S. Ser. No. 13/547,974, entitled “Containment Pant”, filed on Jul. 12, 2012, and having attorney docket number 64816460US01, the entirety of which is incorporated herein by reference.

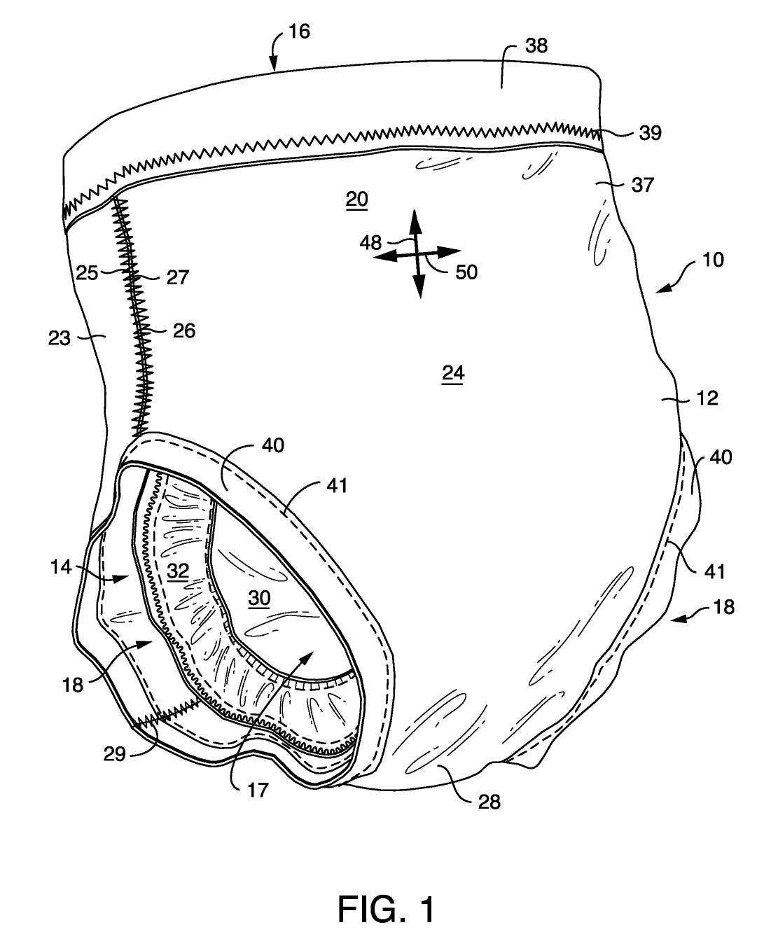

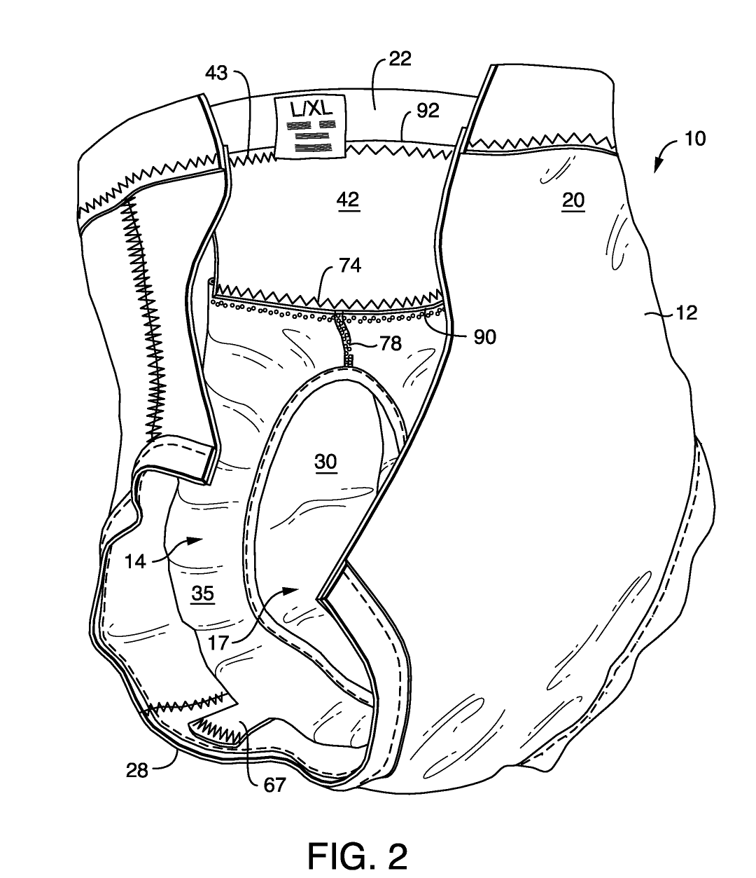

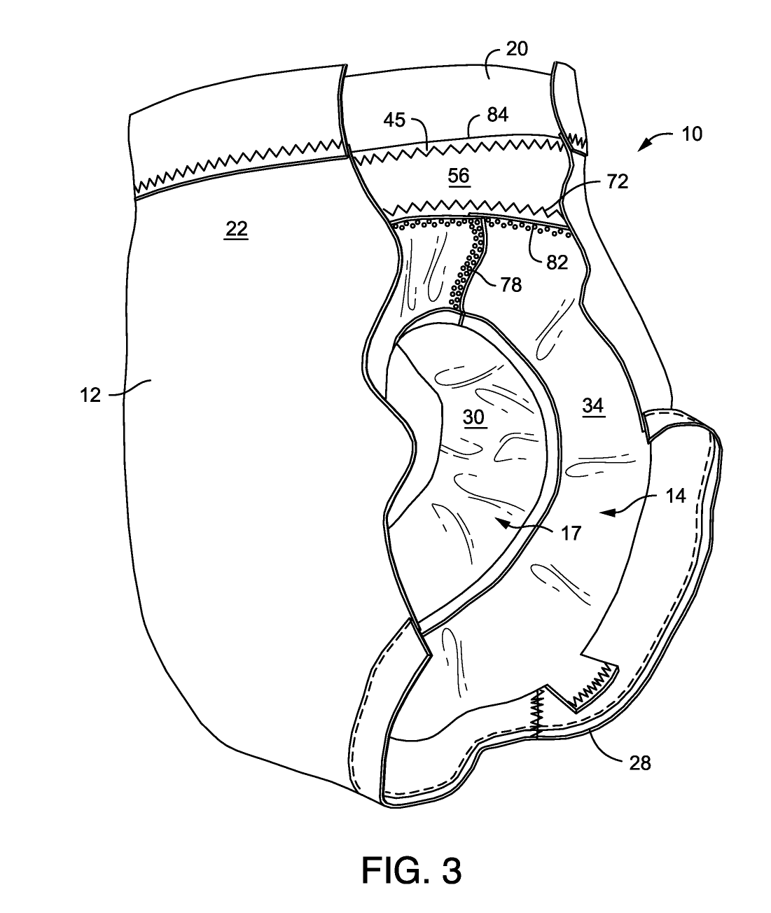

An exemplary containment pant of the present invention is representatively illustrated in FIGS. 1-3. Specifically, FIG. 1 shows a side perspective view of a containment pant 10 having a chassis 12 and a sling 14 attached within the chassis 12. The chassis 12 defines a waist opening 16 and a pair of leg openings 18. The chassis 12 also defines a front waist region 20, a back waist region 22, and a crotch region 28. The crotch region 28 is located between the front waist region 20 and the back waist region 22. The containment pant 10 of FIG. 1 is depicted in FIGS. 2 and 3 with the chassis 12 partially severed to better illustrate internal elements. FIG. 2 representatively illustrates a side perspective view with the chassis 12 partially severed to better illustrate the positioning and construction of the sling 14 in the back of the containment pant 10. FIG. 3 representatively illustrates a side perspective view with the chassis 12 partially severed to better illustrate the construction and positioning of the sling 14 in the front of the containment pant 10. The containment pant 10 defines a longitudinal direction 48 and a transverse direction 50 as illustrated in FIG. 1. The longitudinal direction 48 extends from the front waist region 20 through the crotch region 28 and into the back waist region 22. The transverse direction 50 is perpendicular to the longitudinal direction 48. The chassis 12 includes an outer shell 36 and may further include waist elastic, leg elastic, or both. Referring again to FIG. 1, the chassis 12 is illustrated with a waist elastic 38 attached to the outer shell 36 and encircling the waist opening 16 and leg elastics 40 attached to the outer shell 36 and encircling each of the leg openings 18.

In various embodiments, the outer shell may be made of a single piece of material or multiple pieces of material. In some embodiments, the outer shell may be made of two or more pieces of material.

In various embodiments, the component pieces of the outer shell may be joined together in any suitable manner. For example, the front component 21 may be joined to the back component 23 at a crotch seam. Additionally, one or both of the lateral side edges of the back component may be joined to one or both of the lateral side edges of the front component at one or more side seams to define a three-dimensional garment. Specifically, the lateral side edges 25 of the back component 23 may be joined with the lateral side edges 27 of the front component 21 at side seams 26 to define the leg openings 18 and the waist opening 16 of the containment pant 10 as illustrated in FIG. 1.

In various embodiments, the side seams and/or the crotch seams may be formed using any suitable means such as ultrasonic bonding, thermal bonding, adhesive bonding, pressure bonding, sewing, and the like and combinations thereof. In some embodiments, the side seams and/or the crotch seam may be formed by sewing the component pieces of the outer shell using thread and any suitable stitch pattern or combination of patterns. In some embodiments, the side seams and/or crotch seams may be formed using a flatlock stitch.

In various embodiments, the waist elastic and/or leg elastic may be joined with the outer shell using any suitable means such as ultrasonic bonding, thermal bonding, adhesive bonding, pressure bonding, sewing, and the like and combinations thereof. In some embodiments, the waist elastic and/or leg elastic may be sewed to the outer shell using thread and any suitable stitch pattern or combination of patterns. For example, FIGS. 1-3 representatively illustrate the waist elastic 38 joined with the outer shell 36 at a sewn waist elastic seam 39 and the leg elastics 40 joined with the respective outer shells 36 and 37 at sewn leg elastic seams 41. In some embodiments, the waist elastic and/or leg elastic may be sewed to the outer shell using a cover stitch.

Inside the chassis 12 is the sling 14, which includes a fluid-impervious base sheet 30 and at least first containment flap 32 and second containment flap 34 joined with the base sheet 30 to create a fluid-impervious sling 19. The fluid-impervious sling 19 is adapted to house a removable absorbent insert and contain fluid until it can be taken into the absorbent insert. The fluid-impervious sling 19 is drapeable and is designed to conform and gasket against the body of the wearer.

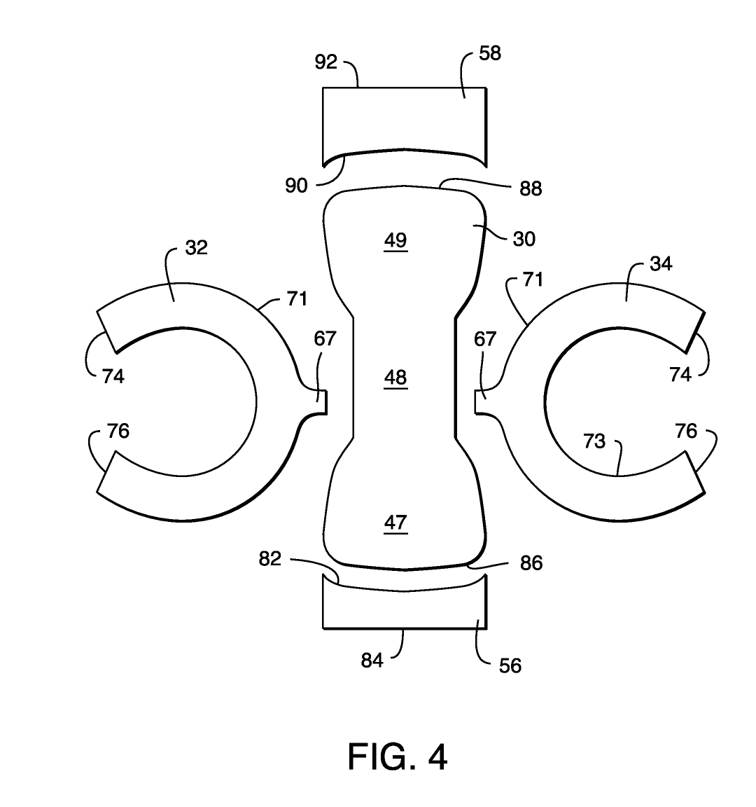

The sling and a fluid-impervious pouch may be constructed with a first containment flap, a second containment flap, a base sheet, a first transition, and a second transition. For example, referring now to FIG. 4, a top plan view of the component parts of another exemplary pouch and sling are representatively illustrated. A fully constructed sling 14 made from the component parts of FIG. 4 is representatively illustrated in FIG. 4. The component parts include a first side containment flap 32, a second side containment flap 34, a base sheet 30, a first transition 56, and a second transition 58. The base sheet 30 may have a curvilinear shape wherein the base sheet defines a first portion 47, a second portion 48, and a third portion 49. In the illustrated embodiment, the first portion 47 has curvilinear side edges and a maximum width that is greater than the maximum width of the second portion 48. Additionally, the third portion 49 has curvilinear side edges and a maximum width that is greater than the maximum width of the second portion 48. The second portion 48 has relatively straight and parallel side edges. The base sheet 30 defines an absorbent facing surface and a chassis facing surface.

In various embodiments, the side containment flaps ideally have a curved cut. For example, the first side containment flap 32 and the second side containment flap 34 of FIG. 4 are illustrated as having a curved cut. While not wishing to be bound by theory, it is believed that the curved cut containment flaps facilitate closer alignment with the natural curvature of the wearer's body. As such, it is believed that a more comfortable and better fit can be achieved to reduce leakage during use.

The first side and second containment flaps 32 and 34 each define a proximal portion 71 and a distal portion 73. A first end 74 of the first containment flap 32 is joined with a first end 74 of the second containment flap 34 to define a first bridging seam 78. Likewise, the second end 76 of the first containment flap 32 and the second end 76 of the second containment flap 34 are joined to define a second bridging seam 78.

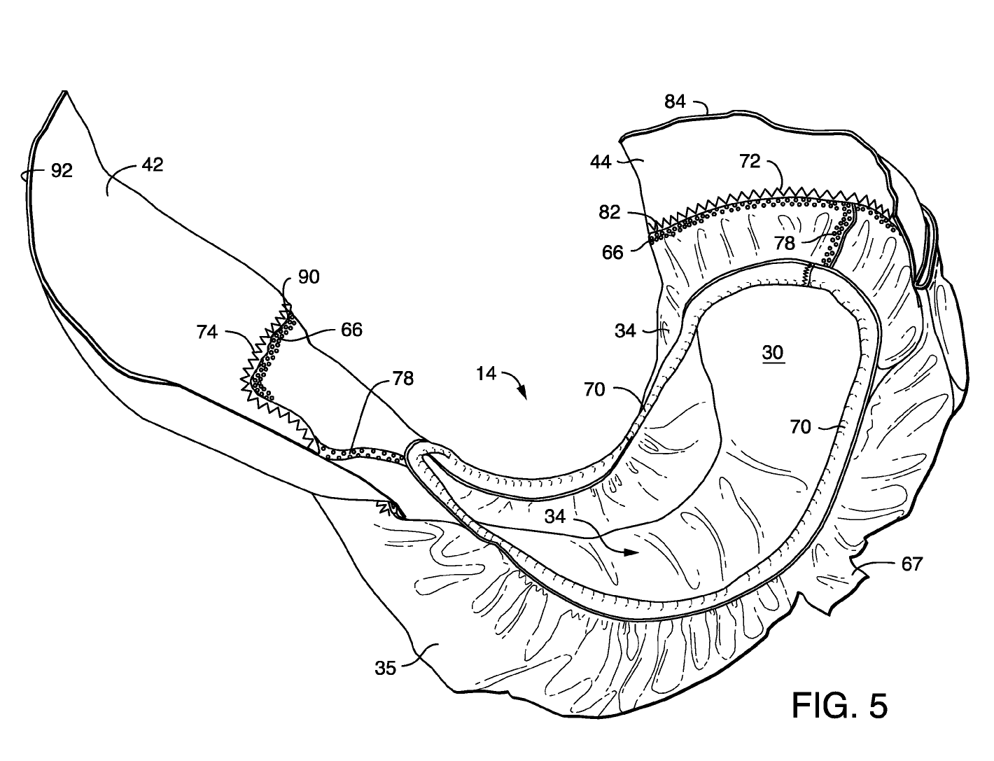

The proximal portions 412 of each containment flap 32 and 34 is joined with the base sheet 30 to create a containment flap seal 466 and define a fluid-impervious pouch 17 as illustrated in FIG. 5. In some embodiments, the containment flaps may further include one or more elastics. For example, as illustrated in FIG. 14, the distal portion 73 of each of the containment flaps 32 and 34 further includes a containment flap elastic 470.

In various embodiments, the fluid-impervious pouch 17 may be incorporated into any suitable sling and/or chassis using integrated transitions, discrete transitions, or combinations thereof. In various embodiments, the fluid-impervious pouch 17 may be incorporated into any suitable sling and/or chassis using only a transition joined with the front waist region, only a transition joined with the back waist region, or neither. In FIG. 5 the fluid-impervious pouch 17 is joined with the first transition 56 and the second transition 58 to define the sling 14. The first transition 56 defines a first edge 82 and a second edge 84. Likewise, the second transition 58 defines a first edge 90 and a second edge 92. The base sheet 30 defines a first edge 86 and a second edge 88. Desirably, the first edge 86 and the second edge 88 of the base sheet 30 define curved convex shapes. The first edge 86 of the base sheet 30 is joined with the first edge 82 of the first transition 56 to define a first junction 72. The second edge 84 of the first transition 56 defines a curved shape to match the first edge of the base sheet 30. Likewise, the second edge 88 of the base sheet 30 is joined with the first edge 90 of the second transition 58 to define a second junction 74. The first edge 90 of the second transition 58 defines a curved shape to match the second edge 88 of the base sheet 30. The first and second junction 72 and 74 may include adhesive bonding, thermal bonding, ultrasonic bonding, pressure bonding, and the like, and combinations thereof.

In this embodiment, the side containment flaps 32 and 34 both include an integral attachment tab 67. The sling 14 may be joined with a chassis in the crotch region 28 using, in part, the attachment tabs 67.

In various embodiments, the sling 14 of FIG. 5 can be joined with any suitable chassis to form a containment pant. In various embodiments, the sling 14 may be joined with a chassis such that the first transition is located in either the front waist region or the back waist region of the chassis and the second transition is located in the opposite region of the chassis. For example, referring now to FIGS. 2 and 3, an exemplary containment pant 10 having a chassis 12 and the sling 14 is representatively illustrated. FIG. 2 is a side perspective view of the containment pant 10 with the chassis 12 partially severed to illustrate the attachment of the sling 14 in the back waist region 22. Specifically, the second transition 58 is joined with the back waist region 22 of the chassis 12 at a back sling seam 43. FIG. 3 is a side perspective view of the containment pant 10 with the chassis 12 partially severed to illustrate the attachment of the sling 14 in the front waist region 20. Specifically, the first transition 56 is joined with the front waist region 20 of the chassis 12 at a front sling seam 45.

In various embodiments, the pouch floor may define any suitable shape. For example, the pouch floor may have a symmetric shape about the transverse centerline or may have an asymmetric shape about the transverse centerline. In various embodiments, the pouch floor may have a dog-bone-shape, hourglass-shape, T-shape, rectangular shape, or the like.

In various embodiments, the pouches of the present invention may be joined with one or more transitions in any suitable manner. For example, the pouches may be joined with the transitions by sewing, ultrasonic bonding, thermal bonding, adhesive bonding, seam taping, and the like, and combinations thereof. In some embodiments, the transitions may be joined with the pouches by sewing, using any suitable stitch or combination of stitches. For example, in some embodiments, the transitions may be attached to the pouches using a single needle stitch followed by a top stitch.

In some embodiments, the pouches may be constructed such that extra material is available for attaching the transitions without breaching the fluid-impervious integrity of the pouch. For example, in some embodiments, the containment flap may be joined to the base sheet at the containment flap seal. The containment flap seal may be positioned such that a portion of the containment flap material and/or the base sheet material is available for attaching to the first and/or second transition while maintaining the integrity of the fluid-impervious pouch.

In some embodiments, the sling is minimally attached to the chassis to maximize the fit and natural movement of the chassis. In some embodiments, the sling is attached to the chassis via the first transition and/or the second transition. In various embodiments, the first transition may be joined with the front waist region at the front sling seam and the second transition may be joined with the back waist region at the back sling seam. In other embodiments, the first transition may be joined with the back waist region at the back sling seam and the second transition may be joined with the front waist region at the front sling seam. The first transition and/or the second transition may be joined to any suitable portion of the front waist region and/or the back waist region. For example, the first transition and/or the second transition may be joined at the waist elastic seam in the front waist region and/or the back waist region. Additionally or alternatively, the first transition and/or the second transition may be joined to the outer shell in the front waist region and/or the second waist region. In some embodiments, the first transition and/or the second transition may be integral with the chassis. In these embodiments, the integral transition is joined with the pouch using any suitable method, including those described herein. In some embodiments, the first transition and/or the second transition may be integral with the shell of the chassis.

In some of the embodiments, the sling may be attached to the chassis in the front waist region, the back waist region, and the crotch region. The sling may be attached to the chassis in the crotch region by any suitable means. For example, the sling may be sewed, ultrasonic bonded, thermal bonded, adhesive bonded, pressure bonded, and the like, and combinations thereof. In some embodiments, the sling may be attached to the chassis in the crotch region via one or more tabs. The tabs may be discrete pieces of material that are joined with both the pouch of the sling and with the chassis using any suitable means. For example, in some embodiments, the tabs may be discrete pieces of material that are ultrasonically bonded to the pouch of the sling and sewn with thread to the chassis. In various embodiments, the tabs may be attached to the chassis using any suitable sewing stitch or combinations thereof. For example, in some embodiments, the tabs may be attached to the chassis using a zig zag stitch.

In various embodiments, at least one of the materials of the containment pant is treated to be more fluid impervious. For example, in any of the embodiments described herein, at least one of the shell, waist elastic, leg elastic, transitions, base sheet, containment flaps, containment flap elastic, and thread are treated to be more fluid impervious. In various embodiments, the treatment may include coating the materials in any suitable manner using a durable water resistant treatment. In some embodiments, the durable water resistant treatment may include a polymer coating. In some embodiments, the base sheet and/or the containment flaps may include a fabric laminated with polyurethane wherein the fabric side of the base sheet is treated with a durable water resistant treatment. In some embodiments, the base sheet and the containment flaps may be made from a woven polyester fabric treated with a durable water resistant treatment and laminated with a polyurethane sheet. In some embodiments, the thread used to join the containment flap elastic to the containment flaps may be treated with a durable water resistant treatment.

In some embodiments, the containment pants of the present invention may be adapted to fit a wide range of sizes. In some embodiments, an exemplary containment pant may be adapted to fit children weighing between 38 and 65 pounds. In some embodiments, an exemplary containment pant may be adapted to fit children weighing between 60 and 120 pounds. To facilitate such a wide range of weight and maintain proper fit, the containment pants of the present invention may be adapted in the waist, hip, and/or legs to extend up to about 100% while still providing sufficient retractive force to hold the pants securely against the body at donning, during use, and after insult.

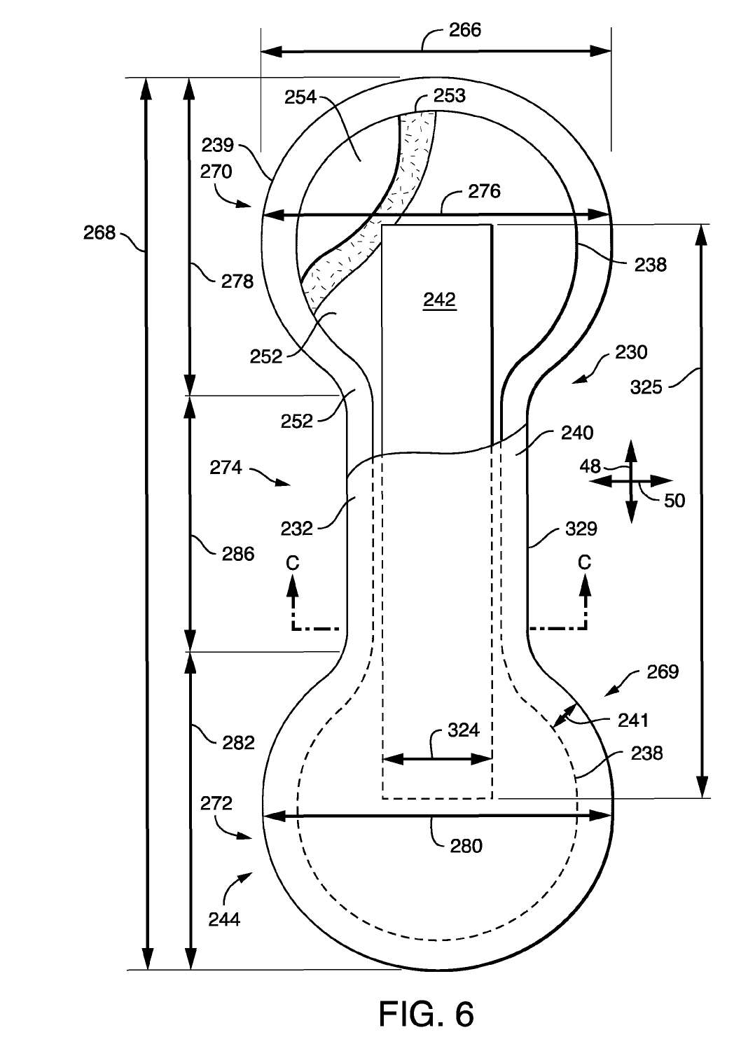

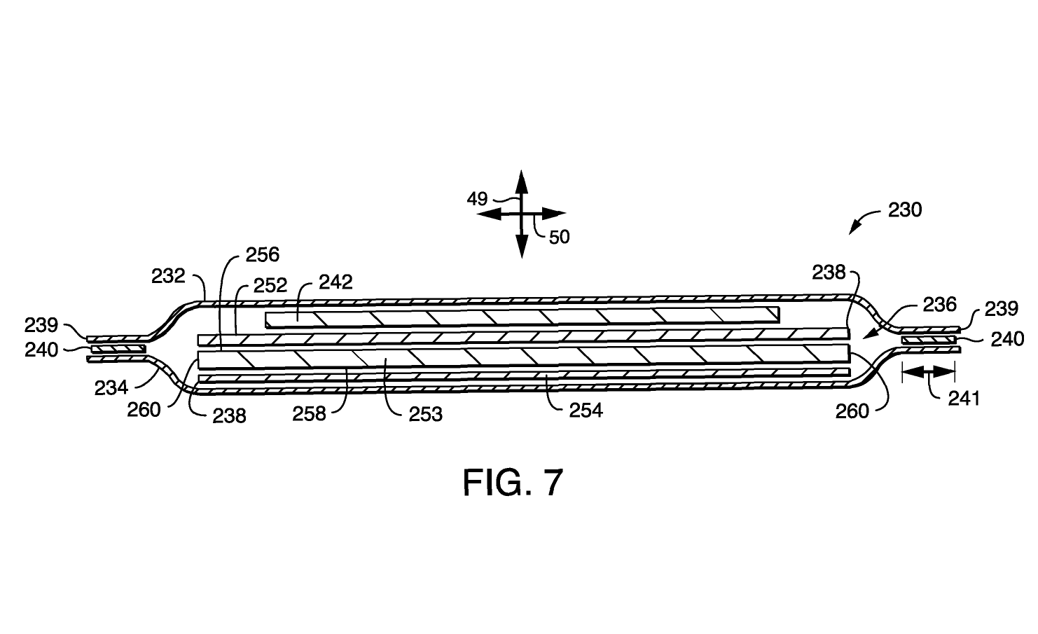

Referring now to FIGS. 35 and 36, a first exemplary absorbent insert 230 is representatively illustrated. FIG. 6 is a top plan view of the absorbent insert 230 with portions cut away to better illustrate underlying structure. FIG. 7 is an expanded cross-sectional view of the absorbent insert of FIG. 6 taken along the line C-C. The absorbent insert 230 defines a longitudinal-direction 48, a relatively shorter, transverse direction 50, and a thickness direction 49. The transverse direction extends generally perpendicular to the longitudinal direction, and the thickness or z-direction extends generally perpendicular to both the longitudinal-direction and transverse direction.

The absorbent insert 230 includes a first sheet 232 and a second sheet 234 in facing relation with the first sheet 232. The absorbent insert 230 also includes an absorbent core 236 positioned between the first sheet 232 and the second sheet 234. The absorbent core 236 defines a core perimeter 238, and the first sheet 232 and the second sheet 234 extend beyond the core perimeter 238, and are joined together to form a perimeter seal 240. The outer extent of the first sheet 232 and/or the second sheet 234 defines the absorbent insert perimeter 239. The absorbent insert perimeter 239 in turn defines an absorbent insert area 269.

The absorbent insert 230 defines an absorbent insert width 266 and an absorbent insert length 268. The absorbent insert 230 defines a first end section 270, a second end section 272, and a central section 274 in the longitudinal direction 48. The central section 274 extends between the first end section 270 and the second end section 272. The first end section 270 defines a first end section width 276 and a first end section length 278. The second end section 272 defines a second end section width 280 and a second end section length 282. The central section 274 defines a central section width 284 and a central section length 286. The first end section length 278 plus the second end section length 282 plus the central section length 286 equals the absorbent insert length 268. The widths of the various sections are measured in the transverse direction 50 and the lengths of the various sections are measured in the longitudinal direction 48.

In an embodiment adapted for use by a child weighing 60 to 120 pounds, the absorbent insert length may be 425 to 475 mm, or 430 to 450 mm, or about 440 mm. In an embodiment adapted for use by a child weighing 38 to 65 pounds, the absorbent insert length may be 375 to 425 mm, 390 to 410 mm, or about 400 mm. In various embodiments, the first end section length may equal the central section length which may equal the second end section length. In other embodiments, the first end section length may equal the second end section length and the central section length may be different. For example, the first end section length may be about 35% the absorbent insert length, the second end section length may be about 35% the absorbent insert length, and the central section length may be about 30% the absorbent insert length. In another example, the first end section length may be about 30% the absorbent insert length, the second end section length may be about 30% the absorbent insert length, and the central section length may be about 40% the absorbent insert length.

To provide an optimal fit within the pouch of the containment pant, the absorbent insert may be shaped to define an arc 290 at both the first end section 270 and second end section 272. In this embodiment, as illustrated in FIG. 6, both the first end section 270 and the second end section 272 define an arc 290 extending at least 185 degrees. In this embodiment, the first end section 270 defines a maximum width of at least 120 mm, the central section defines a maximum width of less than 115 mm, and the second end section 272 defines a maximum width of at least 120 mm. In another embodiment, both the first end section and the second end section define a circular arc 290 extending at least 270 degrees. In this embodiment, the first end section 270 defines a maximum width of at least 150 mm, the central section defines a maximum width of less than 100 mm, and the second end section 272 defines a maximum width of at least 150 mm.

In various embodiments, the absorbent insert may have a width that varies at different points along the longitudinal direction. For example, as illustrated in FIG. 6, the absorbent insert 230 has a variable width at different points along the longitudinal direction. In this embodiment, the first end section 270 and the second end section 272 have a maximum width that is greater than the maximum width of the central section 274. For example, in some embodiments, the first end section and the second end section may have a maximum width of about 160 to 210 mm, 170 to 200 mm, 180 to 190 mm or about 186 mm. In these embodiments, the central section may have a maximum width of about 90 to 130 mm, about 100 to 120 mm, or about 114 mm.

In various embodiments, the first end section, the second end section, and/or the central section may have a variable width at different points along the longitudinal direction or may have a consistent width at different points along the longitudinal direction. In some embodiments, the first end section and the second end section may have variable width at different points along the longitudinal direction and the central section may have a consistent width at different points along the longitudinal direction as illustrated in FIG. 6. For examples, the first end section and the second end section may have a width that varies from 186 mm at the maximum to 114 mm at the minimum. In these embodiments, the central section may have a consistent width of about 114 mm.

The absorbent insert 230 also defines an absorbent insert area 269. In various embodiments, the absorbent insert area 269 may be any suitable value. For example, in embodiments adapted for use by a child weighing 60 to 120 pounds, the absorbent insert area 269 may be about 65,000 mm². In embodiments adapted for use by a child weighing 38 to 65 pounds, the absorbent insert area 269 may be about 57,600 mm².

In an embodiment adapted for use by a child weighing 60 to 120 pounds, the absorbent core length may be 350 to 450 mm, 375 to 425 mm, 390 to 410 mm, or about 400 mm. In an embodiment adapted for use by a child weighing 38 to 65 pounds, the absorbent core length may be 340 to 380 mm, 350 to 370 mm, or about 360 mm. In various embodiments, the first end section length may equal the central section length which may equal the second end section length. In other embodiments, the first end section length may equal the second end section length and the central section length may be different. For example, the first end section length may be about 25% the absorbent core length, the second end section length may be about 25% the absorbent core length, and the central section length may be about 50% the absorbent core length. In another example, the first end section length may be about 30% the absorbent core length, the second end section length may be about 30% the absorbent core length, and the central section length may be about 40% the absorbent core length.

In various embodiments, the first end section, the second end section, and/or the central section may have a variable width at different points along the longitudinal direction or may have a consistent width at different points along the longitudinal direction. In some embodiments, the first end section and the second end section may have variable width at different points along the longitudinal direction and the central section may have a consistent width at different points along the longitudinal direction as illustrated in FIG. 7. In some embodiments, the first end section and the second end section may have a width that varies from 145 mm at the maximum to 75 mm at the minimum. In these embodiments, the central section may have a consistent width of about 75 mm.

The absorbent core also defines an absorbent core area. In various embodiments, the absorbent core area may be any suitable value. For example, in an embodiment adapted for use by a child weighing 60 to 120 pounds, the absorbent core area may be about 42,000 mm². In an embodiment adapted for use by a child weighing 38 to 65 pounds, the absorbent core area may be about 36,300 mm².

In various embodiments, the perimeter seal may have any suitable width and may be formed by any suitable method. Referring again to FIGS. 5-6, the perimeter seal width 241 is representatively illustrated. In some embodiments, the perimeter seal width may be at least 5, at least 10, at least 15, or at least 20 mm. The perimeter seal may include adhesive bonding, thermal bonding, ultrasonic bonding, pressure bonding, and the like, and combinations thereof. In some embodiments, the first sheet may be joined to the second sheet at the perimeter seal via adhesive bonding and the perimeter seal width may be at least 20 mm. In other embodiments, the first sheet may be joined to the second sheet at the perimeter seal via ultrasonic bonding and the perimeter seal width may be at least 10 mm.

In some embodiments, the absorbent insert may be substantially devoid of fluid-impervious materials. In some embodiments, the absorbent insert does not include a fluid-impervious barrier layer. In comparison, many absorbent articles include a fluid-impervious back sheet or baffle which is provided to prevent fluid from contacting the clothes of the wearer or a delay layer which is provided to slow or divert the fluid. In the present invention, the absorbent insert is positioned within the fluid-impervious pouch and thus does not require a fluid-impervious layer as part of the absorbent insert. Additionally, this design is believed to be beneficial in some embodiments over conventional inserts because fluid can be absorbed into the absorbent insert along the entire pad, including the body-facing surface, the garment-facing surface, and the sides. Additionally, the omission of a fluid-impervious layer eliminates the risk of fluid being trapped between the fluid-impervious pouch and the absorbent insert which might cause leaking during use or leaking when removing the absorbent insert from the pouch.

In some embodiments, the absorbent insert may further include one or more intake layers. For example, the absorbent insert 230 of FIGS. 35 and 36 is illustrated with a first intake material 242 positioned between the first sheet 232 and the absorbent core 236. In some embodiments, the absorbent insert may additionally or alternatively include a second intake material.

In various embodiments, the first intake material and/or the second intake material may have any suitable length, width, or shape. For example, referring again to FIG. 6, the first intake material 242 defines an intake material width 324 and an intake material length 325. In various embodiments, the intake material width may be 50 to 70 mm or about 62 mm. In these embodiments, the intake material width may be at least 70%, 80%, or 90% the second reference width 317 of the absorbent core. In some embodiments, the intake material width may be about 84% the second reference width 317 of the absorbent core.

In some embodiments, the intake material length may be about 325 to 375 mm, 340 to 360 mm, or about 355 mm. In these embodiments, the intake material length may be at least 70%, 80%, or 90% the absorbent core length. In some embodiments, the intake material length may be about 88% the absorbent core length.

In various embodiments, the intake materials may be rectangular as illustrated in FIG. 6 or may be any other suitable shape. For example, in various embodiments, the intake materials may be shaped similarly to the absorbent insert and/or the absorbent core.

In various embodiments, any of the absorbent inserts of the present invention may include absorbent cores having absorbent material and one or more wrap sheets. For example, in some embodiments, the absorbent cores may include a single wrap sheet folded around the longitudinal side edges of the absorbent material and overlapping upon itself to form a fully wrapped absorbent core. In other embodiments, the absorbent core may include two wrap sheets. In these embodiments, one of the wrap sheets may be primarily positioned on a first-facing surface of the absorbent material. The other wrap sheet may be primarily positioned on the second-facing surface of the absorbent material. In these embodiments, the wrap sheet on the second-facing surface may extend to the longitudinal side edges of the absorbent material, may wrap around the longitudinal side edges of the absorbent material, or may extend to the first-facing surface of the absorbent material. Likewise, the wrap sheet on the first-facing surface may extend to the longitudinal side edges of the absorbent material, may wrap around the longitudinal side edges of the absorbent material, or may extend to the second-facing surface of the absorbent material. The wrap sheets may overlap themselves or may overlap each other.

Referring again to FIG. 7, the absorbent core 236 is representatively illustrated with a first wrap sheet 252 positioned on a first-facing surface 256 of the absorbent material 253. The first wrap sheet 252 extends between the longitudinal side edges 260 of the absorbent material 253. The absorbent core 236 also includes a second wrap sheet 254 positioned on a second-facing surface 258 of the absorbent material 253. The second wrap sheet 254 extends between the longitudinal side edges 260 of the absorbent material 253.

In various embodiments, the first sheet and the second sheet may be made of the same material or may be different. In some embodiments, the first sheet and the second sheet may have the same basis weight or may be different. The first sheet and/or the second sheet may be fluid permeable and may be made of substantially hydrophobic fibrous material. For example, the first sheet and/or the second sheet may be a spunbond web composed of synthetic polymer filaments. In some embodiments, the first sheet and/or the second sheet may be a meltblown web or a bonded-carded-web composed of synthetic polymer filaments. Suitable synthetic polymers include, for example, polyethylene, polypropylene, polyester, and the like, and combinations thereof. In some embodiments, both the first sheet and the second sheet are spunbond polypropylene nonwoven webs having an individual basis weight of about 15 gsm. In some embodiments, the first sheet and/or the second sheet may be treated with surfactants to adjust the degree of hydrophobicity and wettability. In some embodiments, the first sheet and/or the second sheet may be embossed, apertured, slit, or otherwise mechanically worked.

The absorbent core typically includes absorbent material composed of airlaid, cellulosic fibers commonly referred to as wood pulp fluff. Other natural fibers, such as cotton, may also be employed to form the absorbent core. The absorbent core can have a density ranging from about 0.18-0.30 grams/cc. This density range allows the absorbent core to be sufficiently flexible to readily conform to the body of the wearer yet maintain sufficient rigidity for insertion into the pouch. In some embodiments, the absorbent core may have a density of about 0.24 grams/cc. The absorbent core may alternatively or additionally include a coform material composed of a mixture of cellulosic fibers and synthetic polymer fibers. For example, the coform material may be composed of an airlaid blend of cellulosic fibers and meltblown polyolefin fibers, such as polyethylene and/or polypropylene fibers. In addition, the absorbent core may have a dry thickness of about 1 to 5 mm or about 2 mm, as measured under a restraining pressure of 0.068 psi (0.47 kPa).

The absorbent core may also include an effective amount of an inorganic or organic high-absorbency (e.g., superabsorbent) material to enhance the absorptive capacity of the absorbent body. For example, the absorbent core can contain 5-95 weight percent high-absorbency material, and preferably includes about 30-70, 40-60, or about 50 weight percent of the high-absorbency material to provide more efficient performance. In some embodiments, the absorbent core can include equal amounts of fluff and superabsorbent. For example, in some embodiments, the absorbent core may include at least 14, at least 15, at least 16, at least 17, at least 18, or at least 19 grams of superabsorbent. In some embodiments, the absorbent core may include at least 14, at least 15, at least 16, at least 17, at least 18, or at least 19 grams of fluff. In some embodiments, the absorbent core may include about 19 grams of superabsorbent and about 19 grams of fluff. In other embodiments, the absorbent core may include about 17 grams of superabsorbent and about 17 grams of fluff.

Suitable inorganic high-absorbency materials include, for example, absorbent clays and silica gels. Organic high-absorbency materials can include natural materials, such as agar, pectin, guar gum and peat moss, as well as synthetic materials, such as synthetic hydrogel polymers. Such hydrogel polymers include, for example, carboxymethylcellulose, alkali metal salts of polyacrylic acids, polyacrylamides, polyvinyl ethers, hydroxypropyl cellulose, polyvinyl morpholinone, polymers and copolymers of vinyl sulfonic acid, polyacrylates, polyacrylamides, polyvinyl pyridine and the like. Other suitable polymers include hydrolyzed acrylonitrile grafted starch, acrylic acid grafted starch, and isobutylene maleic anhydride copolymers, and mixtures thereof. The hydrogel polymers are preferably lightly cross-linked to impart desired levels of water insolubility to the material.

In some embodiments, the wrap sheet may be woven or non-woven material and may be made of any suitable material. For example, the wrap sheet may be made of polypropylene, cellulosic tissue, and the like, and combinations thereof. In some embodiments, the wrap sheet may be nonwoven material made from spunbond polypropylene and may have a basis weight of about 10 gsm.

The intake layer or layers help to decelerate and diffuse surges or gushes of fluid that can be rapidly introduced into the absorbent insert. Desirably, the intake layer can rapidly accept and temporarily hold the fluid prior to releasing the fluid into the absorbent core of the absorbent insert. In some embodiments, the intake layer may be a through air bonded carded web composed of 40% hollow polypropylene fibers (6 denier) and 60% bicomponent fibers (6 denier) (bicomponent sheath: polypropylene core). In various embodiments, the intake layer may have any suitable basis weight. For example, the intake layer may have a basis weight of at least 30, at least 50, at least 75, at least 100, or at least 125 grams per square meter (gsm). In some embodiments, the intake layer may have a basis weight of about 128 gsm. Other examples of suitable intake layers are described in U.S. Pat. No. 5,486,166; U.S. Pat. No. 5,490,846; and U.S. Pat. No. 5,820,973. The entire disclosures of these patents are hereby incorporated by reference herein to the extent they are consistent (i.e., not in conflict) herewith.

In some embodiments, the absorbent insert may have a first intake layer and a second intake layer. In some embodiments, the first intake layer may be the same material as the second intake layer or may be different. In some embodiments, the first intake layer may have the same basis weight as the second intake layer or may be different.

In some embodiments, the absorbent inserts of the present invention may be characterized, at least in part, by specific absorbent properties. For example, in some embodiments, the absorbent inserts may have a total absorbent capacity of at least 600 grams, at least 650 grams, at least 700 grams, or at least 750 grams of 0.9% saline solution. In some embodiments, the absorbent inserts may have an absorbent capacity of about 740 grams or about 827 grams. The total absorbent capacity of the absorbent inserts may be determined by using the Retention Capacity Test Method. This test method measures the amount of fluid retained by an absorbent insert under external pressure. An initial weight of the absorbent insert is measured and then the absorbent insert is submerged in a 0.9% saline solution for 20 minutes. After the saturation time, 0.5 psi pressure is applied across the entire absorbent insert for 5 minutes and the excess saline solution is allowed to drain. After the pressure time, the weight of the saturated absorbent insert is measured. The total absorbent capacity is calculated as the saturated weight minus the initial weight.

In some embodiments, the absorbent inserts may be characterized by total absorbent capacity as a ratio of crotch width. In some embodiments, the products designed for wearers having a weight of 60 to 120 pounds may have an absorbent capacity of about 827 grams and a crotch width of about 74 mm. In other embodiments, the products designed for wearers having a weight of 38 to 65 pounds may have an absorbent capacity of about 740 grams and a crotch width of about 74 mm. Thus, in some embodiments, the ratio of total absorbent capacity to crotch width may be at least 9 g/mm, at least 10 g/mm, or at least 11 g/mm.

The absorbent inserts of the present invention may be provided in any suitable manner. For example, the absorbent inserts may be folded, stacked, wrapped, compressed, or the like, and combinations thereof. In some embodiments, the absorbent inserts may be individually wrapped in a wrapper. In some embodiments, the absorbent inserts may be folded one or more times before being placed in a wrapper. In some embodiments, the absorbent inserts may be folded twice before being placed in the wrapper.

In some embodiments, the absorbent inserts of the present invention include a first sheet, a second sheet, an intake layer, and an absorbent core. In these embodiments, the absorbent core may be positioned between the first sheet and the second sheet. Additionally, the intake layer may be positioned between the first sheet and the absorbent core. In this configuration, the first sheet is designated the body side and the second sheet is designated as the garment side of the absorbent insert.

In various embodiments, the absorbent insert may be folded a first time such that a first portion of the body side is in facing relation with a second portion of the body side. In these embodiments, the absorbent insert may be folded a second time such that a third portion of the body side is in facing relation with a first portion of the garment side. In other embodiments, the absorbent insert may be folded a first time such that a first portion of the garment side is in facing relation with a second portion of the garment side. In these embodiments, the absorbent insert may be folded a second time such that a third portion of the garment side is in facing relation with a first portion of the body side.

In some embodiments, the first fold may be positioned such that the absorbent insert is effectively folded into equal halves. In some embodiments, the first fold and the second fold may be positioned such that the absorbent insert is effectively folded into approximately equal thirds.

In various embodiments, the folded absorbent inserts may be individually packaged in any suitable wrapper material. Conventionally, the wrapper consists of one or more layers of a thin sheet or film of thermoplastic material, such as polyethylene, which is folded around the absorbent article and then sealed by the use of heat and/or pressure, ultrasonics, or an adhesive to form a package or pouch. In various embodiments, the wrapper may include films made from poly(vinyl alcohol), polyvinyl acetate, ethylene vinyl alcohol, polyurethane, ethylene methyl acrylate, and ethylene methyl acrylic acid to make them breathable. In some embodiments, the wrapper material may also be a laminate of different materials, such as a film/nonwoven laminate. The package may have a sealed side or edge that is designed to be opened by breaking or tearing the material at or adjacent the seal in order to subsequently remove the absorbent insert. With some package designs, a flap is provided that folds over the pouch opening and may attach to the front of the pouch with adhesive applied between the pouch and flap, or with a piece of adhesive tape. The sides of the flap may be sealed with the sides of the pouch and may be separated prior to removing the absorbent article.

The containment pants and the absorbent inserts of the present invention are adapted to work together as a containment system. In various embodiments, the containment system includes one or more containment pants like those described herein and one or more absorbent inserts like those described herein. Suitable containment systems are also described in patent application U.S. Ser. No. 13/548,012, entitled “Containment System”, filed on Jul. 12, 2012, and having attorney docket number 64816460US03 and patent application U.S. Ser. No. 13/933,235, entitled “Containment System”, filed on Jul. 13, 2013, the entirety of which are incorporated herein by reference.

In use and prior to donning, the absorbent inserts are positioned within the pouch which is suspended within the containment pant. After the absorbent insert is positioned, the containment system is pulled into place like conventional underwear. The containment pant is adapted to hold the absorbent insert in position to accept one or more urine insults from the wearer. The absorbent insert is adapted to rapidly intake and absorb the urine. The pouch is fluid-impervious and is adapted to hold any urine that may not be immediately absorbed by the absorbent insert. In some embodiments, the absorbent insert is fluid permeable on the garment-facing surface and/or on the side edges as well as the body-side surface. As such, any urine retained by the fluid-impervious pouch may be drawn into the absorbent insert via the garment-facing surface and/or the side edges. The used absorbent insert can be removed from the fluid-impervious pouch and the containment pant may be laundered prior to reuse with a new absorbent insert.

In the embodiment described herein, the absorbent inserts are held in place only by the structure of absorbent insert as it interacts with the containment flaps and/or end pockets. The shape described herein provides the fit necessary to be secured within the containment flaps. In some embodiments, the absorbent inserts are held in place only by the structure of the containment flaps and the containment flap elastic and are devoid of fasteners. In some embodiments, the absorbent inserts are devoid of containment flaps. In these embodiments, the containment of the fluid is managed by the containment flaps of the pouch.

In other embodiments, the absorbent inserts of the present invention may be secured in the pouches of the containment pants by any suitable means. For example, the absorbent inserts may be secured in the pouches via buttons, snaps, hook and loop fasteners, pressure sensitive adhesive, clasps, and the like, and combinations thereof. In some embodiments, the absorbent inserts may be additionally or alternatively held in place by the structure of the containment flaps of the fluid-impervious pouch. For example, the containment flaps may include end pockets that help secure a portion of the absorbent insert within the pouches. In some embodiments, the containment flaps may include flap elastic that provides retractive forces that assist in securing the absorbent insert within the pouch.

In various embodiments, the containment system may include a containment pant having a pouch with a pouch floor area, a length, width, and/or shape adapted to accommodate a specific absorbent insert having a complementary absorbent insert and/or absorbent core area, length, width, and/or shape. This complementary area, length, width, and/or shape is believed to improve the absorbent insert placement on the body, to help contain the absorbent insert within the pouch, and to provide a smooth discrete fit and appearance for discretion.

In some embodiments, a containment pant may have a pouch with a pouch floor having a pouch floor length. Likewise, the absorbent insert may have an absorbent insert length and/or an absorbent core length that is less than or equal to the pouch floor length. In some embodiments, the absorbent insert length and/or an absorbent core length may be at least 70%, at least 80%, at least 90%, or at least 95% the pouch floor length. In one embodiment, the absorbent core length may be 91% of the pouch floor length. In these embodiments, the absorbent insert length and/or an absorbent core length may be 100% or less of the pouch floor length. Having an absorbent insert length and/or an absorbent core length to pouch floor length in these ranges is believed to keep the absorbent insert secure within the pouch without excessive bunching or shifting that may adversely affect performance and/or comfort. An absorbent insert shorter than this range would be more likely to shift or breach the pouch gasket which would likely result in leakage. An absorbent insert longer than this range would be more likely to buckle and bunch within the pouch and may result in a poor user experience due to discomfort and/or loss of discretion.

In some embodiments, a containment pant may have a pouch with a pouch floor having a pouch floor maximum width and a pouch floor minimum width wherein the maximum width and the minimum width are different. Likewise, the absorbent insert may have an absorbent insert and/or absorbent core maximum width and an absorbent insert and/or absorbent core minimum width wherein the maximum width and the minimum width are different. In some embodiments, the absorbent insert and/or the absorbent core maximum width is at least 80%, at least 90%, or at least 95% the pouch floor maximum width. In one embodiment, the absorbent core maximum width is 85% of the pouch floor maximum width. In these embodiments, the absorbent insert and/or absorbent core maximum width may be 100% or less of the pouch floor maximum width. In some embodiments, the absorbent insert and/or the absorbent core minimum width is at least 80%, at least 90%, or at least 95% the pouch floor minimum width. In one embodiment, the absorbent core minimum width may be 91% of the pouch floor minimum width. In these embodiments, the absorbent insert and/or the absorbent core minimum width may be 100% or less of the pouch floor minimum width.

In some embodiments, a containment pant may have a pouch with a pouch floor having a pouch floor maximum width in a front portion and/or a back portion and a pouch floor minimum width at a central portion wherein the maximum width and the minimum width are different. Likewise, the absorbent insert and/or absorbent core may have an absorbent insert and/or absorbent core maximum width in a front portion and/or a back portion and an absorbent insert and/or absorbent core minimum width at a central portion wherein the maximum width and the minimum width are different. In some embodiments, the minimum width of the pouch floor may be less than 80%, less than 70%, less than 60%, or about 62% the maximum width of the pouch floor. Similarly, the minimum width of the absorbent insert and/or absorbent core may be less than 80%, less than 70%, less than 60%, or about 62% the maximum width of the absorbent insert and/or absorbent core.

In one embodiment, the present invention is a method for providing a containment system. The method includes providing a permanently closed containment pant having a pouch like those disclosed herein. The pouch defines a pouch floor having a longitudinal direction, a first end section, a second end section, and a central section extending between the first end section and the second end section. The first end section, the second end section, and the central section define equal lengths in the longitudinal direction and together define a pouch floor length. The first end section defines a maximum width of at least 130 mm, at least 140 mm, at least 150 mm, or at least 165 mm, the central section defines a maximum width of less than 110 mm, less than 100 mm, less than 95 mm, and the second end section defines a maximum width of at least 130 mm, at least 140 mm, at least 150 mm, or at least 165 mm. The permanently closed containment pant is substantially devoid of an integrated absorbent core. The method further includes providing a discrete absorbent insert like those disclosed herein. The discrete absorbent insert includes an absorbent core that defines a longitudinal direction, a first end section, a second end section, and a central section. The central section is positioned between the first end section and the second end section. The first end section, the second end section, and a central section define equal lengths in the longitudinal direction. The first end section defines a maximum width of at least 115 mm, at least 125 mm, or at least 135 mm, the central section defines a maximum width of less than 100 mm, less than 90 mm, or less than 85 mm, and the second end section defines a maximum width of at least 115 mm, at least 125 mm, or at least 135 mm. The discrete absorbent insert may be devoid of a fluid impervious layer.

In some embodiments, the containment system of the present invention includes one or more absorbent inserts having a designated body side and a designated garment side. In other words, in some embodiments, the absorbent inserts are designed to be oriented with a particular side towards the user to maximize the performance of the absorbent insert. For example, in some embodiments, the absorbent insert may have a single intake layer located on one side of the absorbent core. In these embodiments, it is desirable for the user to orient the absorbent insert within the pouch such that the intake layer faces the user. In this orientation, the absorbent insert is positioned to intake fluid rapidly through the intake layer and retain the fluid in the absorbent core.

To assist users in properly orienting the absorbent insert in the pouch, various cues may be provided. In some embodiments, the absorbent insert may include a color on one or more of the components. In some embodiments, the absorbent insert may include an intake layer that has a color that is distinguishable from the other components of the absorbent insert. For example, in some embodiments, the intake layer may have a blue color and the surrounding components may have a white color. In these embodiments, the user may be directed to identify the blue intake layer and position it facing the body. In this way, the user has a simple visual cue to quickly, repeatably, and properly orient the absorbent insert within the pouch.

In some embodiments, the absorbent inserts may be provided folded in individual wrappers. In these embodiments, the absorbent inserts may be folded to define a direction of curvature. The absorbent inserts may be bi-folded, tri-folded, or the like. In these embodiments, the absorbent inserts may be folded such that the resulting curvature is directed to the side of the absorbent insert that is desirably oriented towards the wearer. For example, the absorbent insert may have a single intake layer located on one side of the absorbent core. The absorbent insert may be folded such that the absorbent insert is cupped towards the side having the intake layer. In use, this cupped formation naturally fits with the cupped formation of the pouches within the containment pants. As such, the user is cued to position the absorbent insert within the pouch with the intake layer oriented towards the body of the wearer.

While the invention has been described in detail with respect to specific embodiments thereof, it will be appreciated that those skilled in the art, upon attaining understanding of the foregoing will readily appreciate alterations to, variations of, and equivalents to these embodiments. Accordingly, the scope of the present invention should be assessed as that of the appended claims and any equivalents thereto. Additionally, all combinations and/or sub-combinations of the disclosed embodiments, ranges, examples, and alternatives are also contemplated.

1. A discrete absorbent insert comprising,
a first sheet,
a second sheet in facing relation with the first sheet, and
an absorbent core positioned between the first sheet and the second sheet, wherein the absorbent core defines a core perimeter and the first sheet and the second sheet extend beyond the core perimeter and are joined together to form a perimeter seal,
wherein the absorbent core defines a longitudinal direction, a first end section, a second end section, and a central section, wherein the central section is positioned between the first end section and the second end section, wherein both the first end section and the second end section comprises an arc extending at least 185 degrees, and wherein the first end section defines a maximum width of at least 120 mm, the central section defines a maximum width of less than 115 mm, and the second end section defines a maximum width of at least 120 mm.

2. The discrete absorbent insert of claim 1 further comprising a first intake material positioned between the first sheet and the absorbent core.

3. The discrete absorbent insert of claim 2 further comprising a second intake material positioned between the absorbent core and the second sheet.

4. The discrete absorbent insert of claim 1 wherein the absorbent insert has a dry thickness of at least 1 mm, an absorbent capacity of at least 600 g, and a length of 375 mm to 475 mm.

5. The discrete absorbent insert of claim 1 wherein the first sheet is joined with the second sheet at the perimeter seal with adhesive and the perimeter seal has a width of at least 20 mm.

6. The discrete absorbent insert of claim 1 wherein the first sheet is joined with the second sheet at the perimeter seal with an ultrasonic bond and the perimeter seal has a width of at least 10 mm.

7. The discrete absorbent insert of claim 1 wherein the absorbent core has at least 40% superabsorbent.

8. The discrete absorbent insert of claim 1 wherein the absorbent core has at least 15 grams fluff and at least 15 grams superabsorbent.

9. The discrete absorbent insert of claim 1 wherein the intake layer has a basis weight of at least 85 gsm.

10. The discrete absorbent insert of claim 1 wherein the discrete absorbent insert is folded a first time such that a first portion of the first sheet is in facing relation with a second portion of the first sheet and is folded a second time such that a third portion of the first sheet is in facing relation with a first portion of the second sheet.

11. The discrete absorbent insert of claim 1 wherein the discrete absorbent insert is devoid of fasteners and is devoid of containment flaps.

12. The discrete absorbent insert of claim 1 wherein both the first end section and the second end section comprises a circular arc extending at least 270 degrees wherein the first end section defines a maximum width of at least 150 mm, the central section defines a maximum width of less than 100 mm, and the second end section defines a maximum width of at least 150 mm.

13. A containment system comprising,
a permanently closed containment pant comprising:
an elastically extensible chassis defining a waist opening, a first leg opening, and a second leg opening, the waist opening comprising a front waist region joined with a back waist region, and
a sling positioned within the chassis and being joined to the front waist region and the back waist region,
wherein the sling comprises a fluid-impervious base sheet and a containment flap joined with the base sheet to create a fluid-impervious pouch,
wherein the fluid-impervious pouch defines a pouch floor having a longitudinal direction, a first end section, a second end section, and a central section extending between the first end section and the second end section, wherein the first end section, the second end section, and the central section define equal lengths in the longitudinal direction and together define a pouch floor length, and wherein the first end section defines a maximum width of at least 145 mm, the central section defines a maximum width of less than 115 mm, and the second end section defines a maximum width of at least 145 mm; and
a discrete absorbent insert comprising:
a first sheet,
a second sheet in facing relation with the first sheet,
an absorbent core positioned between the first sheet and the second sheet, wherein the absorbent core defines a core perimeter and the first sheet and the second sheet extend beyond the core perimeter and are joined together to form a perimeter seal, and
an intake layer positioned between the first sheet and the absorbent core, wherein the first sheet and the second sheet are fluid permeable,
wherein the absorbent core defines a first end section, a second end section, and a central section, wherein the central section is positioned between the first end section and the second end section, wherein both the first end section and the second end section comprises a circular arc extending at least 185 degrees, and wherein first end section defines a maximum width of at least 120 mm, the central section defines a maximum width of less than 115 mm, and the second end section defines a maximum width of at least 120 mm.

14. The containment system of claim 13 wherein the discrete absorbent insert is folded a first time such that a first portion of the first sheet is in facing relation with a second portion of the first sheet and is folded a second time such that a third portion of the first sheet is in facing relation with a first portion of the second sheet and wherein the twice-folded discrete absorbent insert is individually positioned within a wrapper.

15. The containment system of claim 13 wherein the absorbent core has at least 17 grams fluff and at least 17 grams superabsorbent.

16. The containment system of claim 13 wherein the absorbent core defines an absorbent core length as measured in the longitudinal direction, the first end section defines a first end section length that is 25-30% the absorbent core length, the second end section defines a second end section length that is 25-30% the absorbent core length, and the central section defines a central section length that is 40-50% the absorbent core length, wherein the first end section and the second end section have a variable width at different points along the longitudinal direction and the central section has a consistent width at different points along the longitudinal direction.

17. The discrete absorbent insert of claim 13 wherein both the first end section and the second end section comprises a circular arc extending at least 270 degrees wherein the first end section defines a maximum width of at least 150 mm, the central section defines a maximum width of less than 100 mm, and the second end section defines a maximum width of at least 150 mm.
